SEQUENCE LISTING (7 sequences)

SEQ ID NO: 1 - protein, 220 aa, Solanum lycopersicum
MGRRPARCYRQIKNKPYPKSRFCRGVPDPKIRIYDVGMKKKGVDEFPFCVHLVSWEKENVSSEALEAARIACNKYMTKSAGKDAFHLRVRVHPFHVLRINKMLSCAGADRLQTGMRGAFGKPQGVCARVAIGQVLLSVRCKDGNANHAQEALRRAKFKFPGRQKIIVSRKWGFTKFSRTDYLKYKSENRIVPDGVNAKLLGCHGRLAARQPGRAFLEAAN

SEQ ID NO: 2 - protein, 220 aa, Zea mays
MGRRPARCYRQIKNKPCPKSRYCRGVPDPKIRIYDVGMKRKGVDEFPYCVHLVSWERENVSSEALEAARIVCNKYMTKSAGKDAFHLRVRVHPFHVLRINKMLSCAGADRLQTGMRGAFGKPQGTCARVDIGQVLLSVRCKDNNAAHASEALRRAKFKFPARQKIIESRKWGFTKFSRADYLKYKSEGRIVPDGVNAKLLANHGRLEKRAPGKAFLDAVA

SEQ ID NO: 3 - protein, 222 aa, Oryza sativa
MGRRPARCYRQIKNKPYPKSRYCRGVPDPKIRIYDVGMKKKGVDEFPYCVHLVSWEKENVSSEALEAARIACNKYMTKNAGKDAFHLRVRVHPFHVLRINKMLSCAGADRLQTGMRGAFGKPQGTCARVDIGQVLLSVRCKESNAKHAEEALRRAKFKFPGRQKIIHSRKWGFTKFTREEYVKLKAEGRIMSDGVNAQGSHGRLAKRAPGKAFLAETIQASA

SEQ ID NO: 4 - protein, 221 aa, Saccharomyces cerevisiae
MARRPARCYRYQKNKPYPKSRYNRAVPDSKIRIYDLGKKKATVDEFPLCVHLVSNELEQLSSEALEAARICANKYMTTVSGRDAFHLRVRVHPFHVLRINKMLSCAGADRLQQGMRGAWGKPHGLAARVDIGQIIFSVRTKDSNKDVVVEGLRRARYKFPGQQKIILSKKWGFTNLDRPEYLKKREAGEVKDDGAFVKFLSKKGSLENNIREFPEYFAAQA

SEQ ID NO: 5 - protein, 221 aa, Arabidopsis thaliana [sequence omitted]

SEQ ID NO: 6 - protein, 221 aa, Schizosaccharomyces pombe
MARRPARCYRYCKNKPYPKSRYNRAVPDSKIRIFDLGRKRAGVDEFPLCIHLVSNEYEQITSEALEAARICANKYLVKIGGKDSFHLRVRAHPFHVVRINKMLSCAGADRLQTGMRHAFGKPNGLVARVNIGQILMSVRTKDSSRATAIEALRRCQYKFPGQQRIIVSKKWGFSQYARDEYIEKRSRGEIIPDGCYAKFLNKRGSLQEKLDLFPEASFNLA

SEQ ID NO: 7 - protein, 214 aa, Homo sapiens
MGRRPARCYRYCKNKPYPKSRFCRGVPDAKIRIFDLGRKKAKVDEFPLCGHMVSDEYEQLSSEALEAARICANKYMVKSCGKDGFHIRVRLHPFHVIRINKMLSCAGADRLQTGMRGAFGKPQGTVARVHIGQVIMSIRTKLQNKEHVIEALRRAKFKFPGRQKIHISKKWGFTKFNADEFEDMVAEKRLIPDGCGVKYIPSRGPLDKWRALHS

(19) United States
(12) Patent Application Publication
(10) Pub. No.: US 2008/0184384 A1
(43) Pub. Date: Jul. 31, 2008

(54) QM protein-mediated stress tolerance in transformed eukaryotes

(75) Inventor: Martin B. Dickman, College Station, TX (US)

Correspondence Address: Biotechnology Law Group;c/o Portfolioip, P.O. Box 52050, Minneapolis, MN, 55402, US

(73) Assignee: iDiverse, Inc., Solana Beach, CA (US)

(21) Appl. No.: 11/705,559

(22) Filed: Feb. 10, 2007

Related U.S. Application Data
(60) Provisional application No. 60/772,285, filed on Feb. 11, 2006.

(51) Int. Cl. C12N 15/11; A01H 5/00; C12N 1/19

(52) U.S. Cl. 800/278; 800/298; 800/317.4; 800/317.2; 800/317.3; 800/314; 800/306; 800/313; 800/312; 800/317.1; 800/317; 800/322; 800/323; 800/320; 800/320.1; 800/320.3; 800/320.2; 800/309; 800/307; 800/310; 435/254.2; 435/471

(57) ABSTRACT

Transgenic eukaryotic organisms and cells exhibiting improved resistance to abiotic and biotic stresses, as compared to wild-type varieties of the same organisms and cells, are described, as are methods for making and using the same. Genetic engineering of genes encoding a QM protein, including introduction of QM-encoding transgenes, engineering of regulatory regions for endogenous QM genes, duplication of QM-encoding genes, etc., confers such improved stress resistance in eukaryotic organisms, particularly plants and cell-based systems for the bioproduction of useful compounds, including recombinant proteins, biofuels, food supplements.

### RELATED APPLICATIONS

This application claims the benefit of and priority to U.S. provisional patent application No. 60/772,285, filed 11 Feb. 2006 and entitled, “Stress tolerance Mediated by QM-like Genes, ” which is hereby incorporated by reference in its entirety for all purposes.

### GOVERNMENT INTEREST

Some of the work described herein was supported in part by funding under NIH pilot grant 5P-20-RR017675-03 and NIH grant number P20 RR-017675-02. Accordingly, the U.S. government may have rights in the inventions described and claimed herein.

### TECHNICAL FIELD

This invention relates generally to nucleic acid molecules and products encoded thereby that surprisingly modulate stress resistance in eukaryotic organisms, including fungi, yeast, and plants.

### BACKGROUND OF THE INVENTION

1. Introduction

The following description includes information that may be useful in understanding the present invention. It is not an admission that any such information is prior art, or relevant, to the presently claimed inventions, or that any publication specifically or implicitly referenced is prior art.

2. Background

A. Programmed Cell Death (PCD).

Programmed cell death (“PCD”) and its morphological equivalent, apoptosis, is the active process of genetically controlled cell suicide. PCD has been found to be an intrinsic part of the development, maintenance of cellular homeostasis, and defense against environmental insults, including pathogen attack, in animals. It also plays an essential role in morphogenesis and in development of the immune and nervous systems. Dysregulation of apoptosis, conversely, is involved in the pathogenesis of a number of important diseases in mammals, including cancers, autoimmunity, AIDS, and neurodegenerative disorders.

With recent advances in understanding the complex signaling pathways that induce programmed cell death in animal cells, research has intensified in identifying similar pathways in evolutionarily distant organisms, such as plants. In plants, PCD plays a normal physiological role in a variety of developmental processes, including xylem formation, senescence, sloughing of root cap cells, and embryogenesis (reviewed by Dickman and Reed (2003), Programmed Cell Death In Plants, edited by John Gray, published by Blackwell Publishing, Chapter 2: Paradigms for programmed cell death in animals and plants). Plant cell death also occurs in response to pathogen challenge, as well as in response to abiotic stresses. Recent evidence suggests that plant cell death might be mechanistically similar to animal apoptosis in some cases such as in plant development, disease associated death, and hypersensitive reaction. The dying plant cells appear morphologically similar to apoptotic cells: they form apoptotic bodies; oligonucleosomal cleavage occurs, often with the characteristics of endonucleolytically processed DNA; and terminal deoxynucleotidyl-transferase-mediated UTP end-labeling has been observed.

Despite these similarities between programmed cell death in plants and animals, some aspects of the function and mechanism of PCD in plants may still differ from what is observed in animals. For example, plant cells do not engulf their dead neighbors, and in some cases, the dead plant cells become part of the very architecture of the plant performing crucial functions such as xylem and phloem. Currently, very little is known about the genes and corresponding proteins that control PCD in plants, and few apoptosis-related animal gene (vertebrate or invertebrate) homologues have been found in detected in plants.

Accordingly, given the recognized importance of apoptosis in animals and the importance of PCD in development and pathogen resistance in plants, understanding analogous plant pathways is extremely valuable, and may lead to methods of regulating the pathway and generating transgenic plants harboring cell death modulators that have unique phenotypic characteristics, such as resistance to various biotic and abiotic insults, as well as increased shelf-life of cut plants, fruits, and vegetables.

B. Proline and Reactive Oxygen Species (ROS).

Reactive oxygen species (ROS) are produced by all aerobically respiring cells. ROS can have detrimental effects on cells by oxidizing lipids, proteins, DNA, and carbohydrates, resulting in disease and lethality. It is therefore essential for aerobic organisms to modulate ROS levels and activities in order to protect against toxicity. It has recently been discovered that the α-amino acid proline functions as a potent antioxidant by scavenging intracellular ROS generation in the phytopathogenic fungal pathogen Colletotrichum trifolii (see co-owned PCT application PCT/US2006/004349). Proline's protective role was extended to the budding yeast Saccharomyces cerevisiae, conferring cell survival in the presence of lethal levels of paraquat, a contact herbicide that uncouples electron transport generating lethal levels of superoxide. However, the mechanisms of proline-mediated stress protection and, in particular, the components involved in proline-dependent signal transduction pathways, are still not well understood.

Intracellular proline levels are controlled by a series of metabolic enzymes that mediate proline synthesis and degradation. In S. cerevisiae, two mitochondrial enzymes, including proline dehydrogenase (Put1p) and Δ¹-pyrroline-5-carboxylate (P5C) dehydrogenase (Put2p), mediate the conversion of proline to glutamate in mitochondria. Evidence indicates that these two enzymes also participate in proline-mediated stress responses. Proline accumulation by mutation or disruption of the PUT1 gene results in enhanced freeze tolerance and desiccation stresses than the wild-type strain. In addition, increased intracellular proline levels in a put1 mutant yeast strain have been correlated with higher tolerance to H₂O₂ exposure. Thus, proline appears not only to act as a compatible solute but also to protect cells against damage during oxidative stress. Interestingly, accumulation of the proline catabolic intermediate P5C by disruption of the PUT2 gene triggers intracellular ROS generation, indicating that proline catabolism contributes to intracellular oxidative stress.

A role for proline metabolic enzymes in oxidative stress has also been reported for other organisms. For example, in a human colon cancer cell line, proline dehydrogenase activity was reportedly induced by p53-dependent initiation of apoptosis and catalyzed proline-mediated ROS formation. The antioxidant enzyme Mn-superoxide dismutase effectively inhibited apoptosis induced by proline dehydrogenase activity. In Arabidopsis thaliana, incompatible interactions with Pseudomonas syringae pv. tomato, which triggers high levels of ROS generation, has also been reported to result in proline accumulation and transcriptional activation of AtP5CS, an enzyme involved in proline biosynthesis. It still remains unclear, however, how these proline metabolic enzymes are regulated in response to oxidative stress.

C. QM Protein.

The small, basic QM protein was first identified as a putative tumor suppressor from the Wilms' tumor cell line. It is highly conserved in species ranging from mammals, plants, worms, insects, and yeasts. The protein is about 24 kilodaltons (kD) in size, and peripherally associates with ribosomes. In humans, this protein contains 213 amino acids. QM appears to be a key regulator for signaling pathways involving SH3 domain-containing membrane proteins (e.g., Src family kinases) since the QM protein has been reported to directly interact with the SH3 domain of Src and c-Yes. Indeed, two different regions of QM reportedly associate with SH3 domains. Moreover, it has been demonstrated that the yeast QM homologue GRC5 is involved in translational control of gene expression in S. cerevisiae based on the observation that GRC5 directly participates in the recombination of 60 and 40 S ribosomal protein subunits. Several QM homologues have been identified in plants but their physiological functions have not yet been described. Synonyms for QM include GRC5, 60S ribosomal protein L10 (RPL10), GRC5, L9, QSR1, Ubiquinol-cytochrome C reductase complex subunit VI-requiring protein, and YLR075W.

3. Definitions

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings.

An “abiotic” insult or stress refers to a plant challenge caused by exposure to a non-viable or non-living agent (i.e., an abiotic agent). Examples of abiotic agents that can cause an abiotic stress include environmental factors such as low moisture (drought), high moisture (flooding), nutrient deficiency, radiation levels, air pollution (ozone, acid rain, sulfur dioxide, etc.), high temperature (hot extremes or heat shock), low temperature (cold extremes or cold shock), and soil toxicity (e.g., toxic levels of salt, heavy metals, etc.), as well as herbicide damage, pesticide damage, or other agricultural practices (e.g., over-fertilization, improper use of chemical sprays, etc.).

“Bioproduction” refers to processes for producing a desired compound, for example, a recombinant protein, alcohol, or a biofuel, in a biological system.

A “biotic” insult or stress refers to a plant challenge caused by viable or biologic agents (i.e., biotic agents). Examples of biotic agents that can cause a biotic stress include insects, fungi, bacteria, viruses, nematodes, viroids, mycloplasmas, etc.

A “host cell” refers to a cell that contains a vector according to the invention.

The terms “include”, “including”, and the like mean “including, without limitation”.

An “isolated nucleic acid molecule” refers to a polynucleotide molecule in the form of a separate fragment or as a component of a larger nucleic acid construct, that has been separated from its source cell (including the chromosome it normally resides in) at least once, and preferably in a substantially pure form. Nucleic acid molecules may be comprised of a wide variety of nucleotides, including deoxyribonucleotides, ribonucleotides, nucleotide analogues in which the pyrimidine or purine base differs from a base that occurs in nature (e.g., adenine, guanine, thymine, cytosine, and uracil) or in which the backbone chemistry linking the various monomers (or dimers or other polymers) differs from the phosphodiester backbone of nucleic acids found in nature, or a combination thereof.

The term “modulate” refers to the ability to alter from a basal level. As used in the context of apoptosis (e.g., to “modulate” apoptosis or PCD), “modulate” refers to the ability to alter or change any biochemical, physiological, or morphological event associated with apoptosis from its basal level. For example, apoptosis has been “modulated” if there has been an alteration in expression of a gene involved in an apoptotic pathway, the interaction of an apoptotic pathway protein with other proteins, the formation of apoptotic bodies, or the DNA cleavage is altered from its original state. Similarly, response to a stress has been “modulated” if, for example, a biochemical, physiological, or morphological parameter (e.g., growth, viability, fruit or send production, photosynthetic rate, rate of respiration or transpiration, etc.) being assessed differs from the level of that parameter in the absence of the stress.

A “patentable” composition (including plants, plants cells, plant tissues, seeds, protoplasts, etc.), process (or method), machine, or article of manufacture according to the invention means that the subject matter satisfies all statutory requirements for patentability in the particular jurisdiction at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically exclude the unpatentable embodiment(s). Also, the claims appended hereto are to be interpreted both to provide the broadest reasonable scope, as well as to preserve their validity. Furthermore, if one or more of the statutory requirements for patentability are amended or if the standards change for assessing whether a particular statutory requirement for patentability is satisfied from the time this application is filed or issues as a patent to a time the validity of one or more of the appended claims is questioned, the claims are to be interpreted in a way that (1) preserves their validity and (2) provides the broadest reasonable interpretation under the circumstances.

A “plant pathogen” refers to any agent that causes a disease state in a plant, including viruses, fungi, bacteria, nematodes, and other microorganisms.

A “plant” refers to a whole plant, including a plantlet. Suitable plants for use in the invention include any plant amenable to techniques that result in the introduction of nucleic acid into a plant cell, including both dicotyledonous and monocotyledonous plants. Representative examples of dicotyledonous plants include tomato, potato, arabidopsis, tobacco, cotton, rapeseed, field beans, soybeans, peppers, lettuce, peas, alfalfa, clover, cole crops or Brassica oleracea (e.g., cabbage, broccoli, cauliflower, and Brussels sprouts), radish, carrot, beets, eggplant, spinach, cucumber, squash, melons, cantaloupe, sunflowers, and various ornamentals. Representative examples of monocotyledonous plants include asparagus, field and sweet corn, barley, wheat, rice, sorghum, onion, pearl millet, rye and oat, and ornamentals.

The term “plant cell” refers to a cell from, or derived from, a plant, including gamete-producing cells and cells (e.g., protoplasts) which are capable of regenerating into whole plants. When a cell has been transformed with a nucleic acid or vector according to the invention, it is host cell.

The term “plant tissue” includes differentiated and undifferentiated tissues of a plant, including roots, stems, shoots, leaves, pollen, seeds, tumor tissue, and various forms of cells in culture, including cell suspensions, protoplasts, embryos, and callus tissue.

A “plurality” means more than one.

The term “operably associated” refers to a functional association, or linkage, between a promoter and a structural gene the expression of which is regulated by the promoter. A “structural gene” refers to a DNA sequence (when integrated or otherwise inserted into a chromosome or other DNA molecule of a host cell that is capable of being replicated and segregated during cell division) that is transcribed into RNA. In the context of this invention, RNA transcribed from, for example, a nucleic acid molecule having a nucleotide sequence according to SEQ ID NO: 1 or 2, is not translated, or used by ribosome as a template for the directing the polymerization of amino acids to form a peptide or polypeptide. In this specification, unless the context otherwise requires, the term “expression”, generally refers to the enzyme-mediated transcription of a DNA molecule into an RNA molecule.

A “promoter” refers to a polynucleotide that directs the transcription of a gene operably associated therewith. Typically a promoter is located in the 5′ region of a gene, proximal to the transcriptional start site of a structural gene. A promoter is functional in plant cells if it is able to direct expression of a gene in plant cells. A promoter is constitutive if it directs transcription of a gene under most environmental conditions and states of development or cell differentiation. A promoter is inducible if it is capable of directly or indirectly activating transcription of a nucleic acid sequence in response to an inducer. A tissue-specific promoter is a promoter that directs transcription of a gene in a specific plant tissue or tissues. An event specific promoter is a promoter that is active or up-regulated only upon the occurrence of an event, such as tumorigenicity or viral infection.

The term “transgene” or “heterologous nucleic acid molecule” refers to a nucleic acid molecule containing at least one structural gene. A heterologous nucleic acid molecule generally, although not necessarily, is a nucleic acid molecule isolated from another species. As will be appreciated, the term “transgene” includes a nucleic acid molecule from the same species, where such molecule has been modified or been placed in operable association with on or more regulatory elements (e.g., a promoter) that differs from the natural or wild-type promoter with which the gene is associated in nature.

A “vector” refers to a DNA or RNA molecule such as a plasmid, cosmid, bacteriophage, or other viral genome that has the capability of replicating in a host cell, and include cloning vectors, shuttle vectors, and expression vectors. A “cloning” or “shuttle” vector typically contains one or several restriction endonuclease recognition sites into which foreign or heterologous DNA molecules can be inserted in a determinable fashion without loss of essential biological function of the vector, as well as a marker gene that encodes a gene product useful for the identification and selection of cells transformed with the vector. An “expression vector” is typically a DNA molecule (although RNA viral genomes may also be used) that includes at least one gene the expression of which is desired in a host cell. Typically, the expression of the gene(s) introduced into the vector for expression is under the control of one or more regulatory elements suitable for use in the intended host cell. Such regulatory elements include enhancers, promoters, termination signals, and polyadenylation sites.

A “wild-type” organism or cell, such as a plant, plant variety, or yeast or other eukaryotic cell (e.g., an insect or mammalian cell used for the expression of a recombinant protein) refers to an organism or cell that has not been genetically modified in accordance with the invention. As such, the organism or cell may, in fact, be genetically engineered, although the engineering contained in such “wild-type” organism or cell relates to a genetic modification (e.g., engineering to express an herbicide resistance gene) unrelated to QM gene engineering.

### SUMMARY OF THE INVENTION

The present invention concerns patentable transgenic eukaryotic organisms, particularly plants and plant cells, tissues, and products, as well as yeasts and cell lines used for bioproduction of desired products, that have been genetically engineered with respect to the expression of a QM gene, derivative, or variant.

Thus, in one aspect, the invention concerns transgenic organisms, particularly plants and plant cells, tissues, and products, as well as yeasts and cell lines used for bioproduction of desired products, that exhibit altered patterns of QM gene expression due to genetic manipulation, as compared to wild-type cells. Such manipulation includes insertion of an expression cassette comprising a QM gene, derivative, or variant and a promoter. In other embodiments, the genetic manipulation concerns placing expression of an endogenous QM gene under the control of different regulatory elements, such as a stress-inducible promoter. In some embodiments, the expression cassette may further comprise a second nucleic acid molecule that encodes an expression product that confers a second desired trait, such as resistance to an insect pest (as may be achieved, for example, by the expression in the plant, or selected cells or tissues thereof, of a toxin that kills an insect pest that preys upon the particular plant species) and/or resistance to an herbicide (for example, glyphosate). Alternatively, the second nucleic acid molecule may encode a desired product, such as a protein (e.g., an antibody, a growth factor, a hormone, an enzyme). The transformed eukaryotic organism or cell may also be genetically modified in any other suitable way.

As a result of such genetic manipulation, the transgenic organism or cells exhibit improved resistance or tolerance to abiotic and biotic stresses, particularly those characterized by increased amounts of ROS, as compared to a wild-type variety of the transgenic organism or cells. Thus, a related aspect concerns transgenic organisms, particularly plants and plant cells, tissues, and products, as well as yeasts and cell lines used for bioproduction of desired products, that exhibit increased stress resistance, or tolerance, to a range of abiotic and biotic stresses as compared to wild type varieties of the same organisms.

The invention also concerns various methods, including those for producing transgenic eukaryotic organisms and cells that have a QM gene-related genetic alteration (e.g., introduction of a foreign QM gene, duplication of an endogenous QM protein-encoding gene, alteration of the regulatory sequence(s) of an endogenous QM protein-encoding gene, etc.), for example, by transforming a eukaryotic cell with a nucleic acid molecule encoding a QM protein, derivative, or variant. Transgenic organisms, such as transgenic plants, may then be generated from such cells. The resulting organisms may then be cultivated. Given their improved stress resistance or tolerance, the transgenic organism and cells of the invention can be cultivated environments where they may be exposed to one or more stresses that, in the absence of expression of a nucleic acid molecule of the invention, would result in injury to or death of the transgenic organism or cells.

These and other aspects and embodiments will be apparent to those skilled in the art upon consideration of this specification.

### BRIEF DESCRIPTION OF THE DRAWINGS

FIG. 1(A) shows the amino acid sequence (SEQ ID NO: 1) of a QM protein from the tomato Solanum lycopersicum. The protein is 220 amino acid residues in length.

FIG. 1(B) shows the amino acid sequence (SEQ ID NO: 2) of a QM protein from Zea mays. The protein is 220 amino acid residues in length, and has a molecular weight of 24,919 Da.

FIG. 1(C) shows the amino acid sequence (SEQ ID NO: 3) of a QM protein from Oryza sativa. The protein is 224 amino acid residues in length.

FIG. 1(D) shows the amino acid sequence (SEQ ID NO: 4) of a QM protein from the yeast Saccharomyces cerevisiae. The protein is 221 amino acid residues in length, and has a molecular weight of 25,361 Da. In the region from residues 160-215, there are two repeats of about 15-17 amino acid residues (from positions 167-181 and 200-216).

FIG. 1(E) shows the amino acid sequence (SEQ ID NO: 5) of a QM protein from Arabidopis thaliana. The protein is 221 amino acid residues in length, and has a molecular weight of 24,909 Da.

FIG. 1(F) shows the amino acid sequence (SEQ ID NO: 6) of a QM protein from the yeast Schizosaccharomyces pombe. The protein is 221 amino acid residues in length, and has a molecular weight of 25,361 Da.

FIG. 1(G) shows the amino acid sequence (SEQ ID NO: 7) of a human QM protein. The protein is 214 amino acid residues in length.

### DETAILED DESCRIPTION

It has been discovered that yeast strains overexpressing Put1p exhibit increased sensitivity to oxidative stress, and are characterized by lower proline and higher ROS levels relative to wild-type yeast. A conditional life/death screen in S. cerevisiae was used to identify plant death-suppressors involved in proline-mediated oxidative stress response. Using this screen, a tomato QM-like protein (tQM) was found to be cytoprotective and significantly reduced levels of intracellular ROS under various lethal oxidative stresses. Moreover, tQM effectively rescued the hypersensitivity of the yeast Put1p-overexpressing strain to oxidants. Genetic analysis revealed that tQM directly interacts with the yeast Put1p protein. While not wishing to be bound by any particular theory, these results indicate that tQM functionally regulates ROS levels by modulating intracellular proline levels. In addition, it was also discovered that mitochondria-mediated yeast lethality caused by over-expression of the mammalian proapoptotic Bax protein could also be rescued by tQM expression, through the inhibition of ROS generation. Thus, engineered QM protein expression can be used to regulate cellular oxidative stress responses by modulating proline-mediated protection.

Accordingly, engineered expression of a QM protein, derivative, variant in transgenic eukaryotic organisms, tissues, and cells can confer resistance or tolerance to a number of abiotic and biotic stresses. In some preferred embodiments, the eukaryotic organisms are plants that have been genetically modified to exhibit an altered pattern of QM expression. In other preferred embodiments, the eukaryotic organisms are cells used for bioproduction of desired products, such as recombinant proteins or other compounds, including alcohol or biofuels. Altered patterns of QM protein expression can be achieved by introducing a nucleic acid molecule encoding a desired QM protein into the eukaryotic organism to be modified in a manner that allows the QM protein to be expressed so as to confer stress protection (i.e., cytoprotection). This can also be achieved by altering the expression pattern of an endogenous QM gene, for example, by placing an QM gene under the control of a stress-inducible promoter. Combinations of altered endogenous QM protein expression and expression of a foreign QM-protein encoding gene can also be used. Nucleic acid molecules encoding QM proteins, vectors (particularly vectors that contain expression cassettes designed to provide for expression of such nucleic acids in transformed eukaryotic organisms), eukaryotic organisms (e.g., plants, as well as yeast and insect and mammalian cell lines useful for bioproduction) transformed with such nucleic acids, and methods for making and using the same, are described in detail, below.

### 1. Nucleic Acids

The nucleic acid molecules of the invention are those that encode a QM protein. Any gene that encodes a QM protein, derivative, or variant can be used. Such genes include those found in nature, as well as those that are whole or partially synthetic. As those in the art will appreciate, once an amino acid sequence of a particular QM protein, derivative, or variant is known, any number of nucleic acid molecules encoding it can be prepared, either from naturally occurring sources or synthetically, based on the degeneracy of the genetic code. If desired, nucleic acid molecules encoding a QM protein can be optimized for expression in a particular organism, for example, by using codons found predominantly in highly expressed proteins that occur naturally in the eukaryote into which the nucleic acid is to be introduced. Such genes may encode a QM protein having a naturally occurring amino acid sequence, as well as proteins that are derivatives or variants. Broadly speaking, a QM protein useful in the practice of the invention refers to any protein having an amino acid sequence of a naturally occurring QM protein, as well as any naturally occurring or non-naturally occurring derivative variant of a QM protein, provided that the derivative or variant confers stress protection to a eukaryotic organism or cell into which is has been introduced by any suitable method. A yeast-based screen for assessing stress protection is described below.

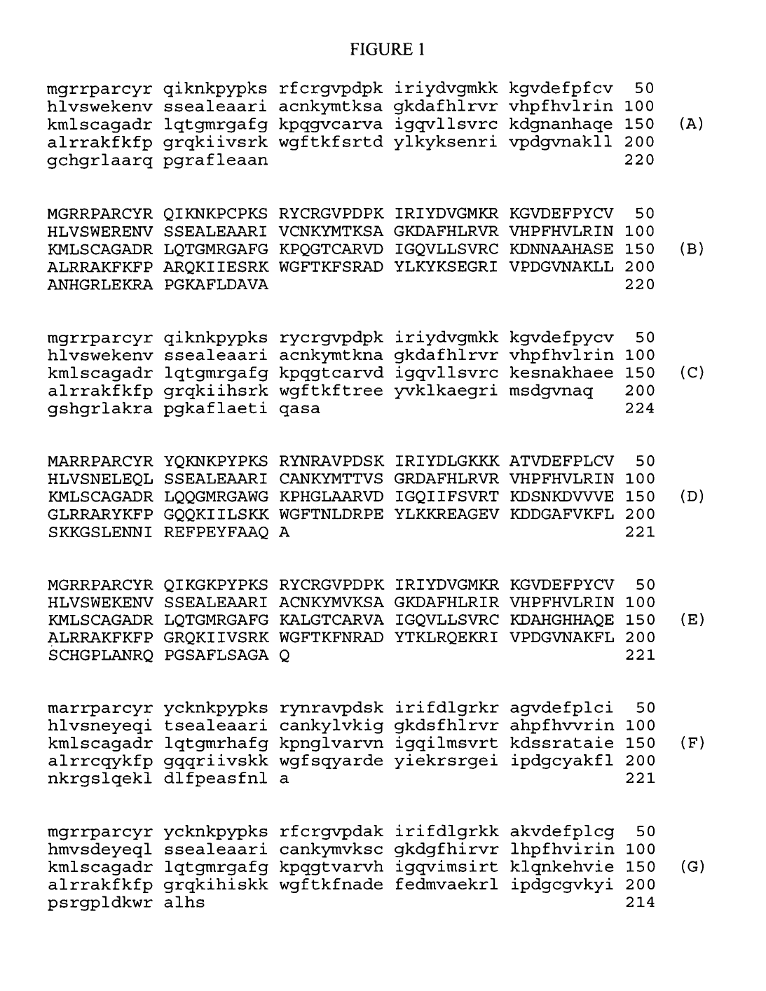

FIG. 1(A-G) lists the amino acid sequences (SEQ ID NOS: 1-7, respectively) of several representative examples of naturally occurring QM proteins useful in conferring stress protection to eukaryotic organisms or cells when expressed from a transgene encoding the same. The nucleic acid molecules can be single- or double-stranded. For purposes of this invention, a gene encoding a QM protein minimally includes polymerized nucleotides encoding a QM protein having the amino acid sequence corresponding to the particular QM protein (or derivative or variant).

Nucleic acids according to the invention or fragments thereof (including those made by various synthetic techniques) may be used as probes for screening to confirm transformation, determine copy number or level of expression of the transgene, etc. To facilitate hybridization-based detection, such probes may be labeled with a reporter molecule, such as a radionuclide (e.g., ³²P, ³⁵S, etc.), enzymatic label, protein label, fluorescent label, biotin, or other detectable moiety. Alternatively, nucleic acid amplification-based techniques known in the art (e.g., PCR, transcription-mediated amplification, strand-displacement amplification, etc.) may be readily adapted for such purposes through the design and use of suitable primers.

### 2. Vectors, Host Cells, and Transgene Expression

The present invention encompasses vectors comprising regulatory elements operably associated with a nucleic acid molecule of the invention, i.e., a nucleic acid having a nucleobase (or nucleotide) sequence that encodes a particular QM protein, derivative, or variant. Such vectors may be used, for example, in the propagation and maintenance of nucleic acid molecules of the invention, or in the expression and production of RNA transcripts and protein from such nucleic acid molecules. Depending upon the intended use, those skilled in the art can select any suitable vector. Suitable vectors include plasmids, cosmids, episomes, and viral genomes, including those adapted for gene transfer from baculovirus, retrovirus, lentivirus, adenovirus, and parvovirus.

Nucleic acid molecules of the invention may be expressed in a variety of host organisms, including mammalian cells (e.g., CHO, COS-7, and 293 cells), other eukaryotes such as yeast (e.g., Saccharomyces cerevisiae) and insect cells (e.g., Sf9), as well as bacterial cells (e.g., E. coli and Bacillus). Expression of a nucleic acid encoding a QM protein, derivative, or variant in, for example, bioproduction, examples of which include the production of a recombinant protein (e.g., an antibody, a growth factor, a hormone, an enzyme, etc.) in a mammalian or insect cell line. Given the stress tolerance conferred by expression of the QM protein, such systems can be used to increase the yield of the desired protein product. The same situation also obtains in the context of producing alcohol in yeast transformed with an expression cassette that directs the expression of a QM protein. In other particularly preferred embodiments, a nucleic acid molecule according to the invention is expressed in plant cells. Vectors suitable for use with any of these host cells are well known in the art.

In preferred embodiments, a DNA molecule encoding a desired QM protein is introduced into a vector to form an expression cassette. The DNA molecule can be derived from an existing clone or synthesized. Preferred synthetic routes include nucleic acid-based amplification (e.g., PCR) of a structural gene of the invention. Such gene may be present, for example, in cDNA, genomic DNA, or in a recombinant clone. Amplification is performed using a set of primers that flank the structural gene. Restriction sites are typically incorporated into the primer molecules to facilitate subsequent cloning steps, and should be chosen with regard to the cloning site of the vector. If desired, termination signals, polyadenylation signals, etc. can also be engineered into an amplification primer.

At minimum, the expression cassette vector will also contain a promoter. The promoter will contain an RNA polymerase binding site, and, in eukaryotes, promoters frequently contain binding sites for other transcriptional factors that control the rate and timing of gene expression. Such sites include the so-called TATA box, CAAT box, POU box, API binding site, and the like. Promoter regions may also contain enhancer elements. The promoter may be in the form of a promoter that is naturally associated with a QM gene. Alternatively, and preferably, the nucleic acid is under control of a promoter other than a QM gene promoter. Such alternative promoters may provide for constitutive or inducible expression of the nucleic acid molecule of the invention, as desired in the particular system.

The expression cassettes of the expression vectors of the invention include a promoter designed for expression of a structural gene according to the invention. Such promoters for expression in bacteria include promoters from the T7 phage and other phages, such as T3, T5, and SP6, and the trp, 1pp, and lac operons. Hybrid promoters (see, e.g., U.S. Pat. No. 4,551,433), such as tac and trc, may also be used. Promoters for expression in eukaryotic cells include the P10 or polyhedron gene promoter of baculovirus/insect cell expression systems (see, e.g., U.S. Pat. Nos. 5,243,041, 5,242,687, 5,266,317, 4,745,051, and 5,169,784), MMTV LTR, CMV IE promoter, RSV LTR, SV40, metallothionein promoter (see, e.g., U.S. U.S. Pat. No. 4,870,009), 35S promoter of CaMV, alcohol dehydrogenase gene promoter, chitinase gene promoter, and the like.

The promoter that controls transcription of a QM protein-encoding gene may itself be controlled by a repressor. In some systems, the promoter can be derepressed by altering the physiological conditions of the cell, for example, by the addition of a molecule that competitively binds the repressor, or by altering the temperature of the growth media. Preferred repressors include the E. coli lacI repressor responsive to IPTG induction, the temperature sensitive lambda cI857 repressor, and the like.

In other preferred embodiments, the vector also includes a transcription terminator sequence. A “transcription terminator region” has either a sequence that provides a signal that terminates transcription by the RNA polymerase that recognizes the selected promoter and/or a signal sequence for polyadenylation.

Preferably, the vector is capable of replication in the host cells. Thus, when the host cell is a bacterium, the vector preferably contains a bacterial origin of replication. Preferred bacterial origins of replication include the fl-ori and col E1 origins of replication, especially the ori derived from pUC plasmids. In yeast, ARS or CEN sequences can be used to assure replication. A well-used system in mammalian cells is SV40 ori.

The plasmids also preferably include at least one selectable marker that is functional in the host cell into which the vector is introduced. A selectable marker gene includes any gene that confers a phenotype on the host that allows transformed cells to be identified and selectively grown. Suitable selectable marker genes for bacterial hosts include the ampicillin resistance gene (Amp^{r}), tetracycline resistance gene (Tc^{r}), and the kanamycin resistance gene (Kan^{r}). The kanamycin resistance gene is presently preferred. Suitable markers for eukaryotes usually require a complementary deficiency in the host (e.g., thymidine kinase (tk) in tk-hosts). However, drug markers are also available (e.g., G418 resistance and hygromycin resistance).

One skilled in the art appreciates that there are a wide variety of suitable vectors for expression in bacterial cells that are readily obtainable. Vectors such as the pET series (Novagen, Madison, Wis.), the tac and trc series (Pharmacia, Uppsala, Sweden), pTTQ18 (Amersham International plc, England), pACYC 177, the pGEX series, and the like are suitable for expression of BAG-1. Baculovirus vectors, such as pBlueBac (see, e.g., U.S. Pat. Nos. 5,278,050, 5,244,805, 5,243,041, 5,242,687, 5,266,317, 4,745,051, and 5,169,784; available from Invitrogen, San Diego) may be used for expression in insect cells, such as Spodoptera frugiperda sf9 cells (see, e.g., U.S. Pat. No. 4,745,051). As will be appreciated, different vectors are paired with suitable hosts.

A wide variety of suitable vectors for expression in eukaryotic cells are also available. Such vectors include pMC1 neo, pOG, pCMVLacI, and pXT1 series vectors available from Stratagene Cloning Systems (La Jolla, Calif.), and pCDNA series, pREP series, and pEBVHis available from Invitrogen (Carlsbad, Calif.). In certain embodiments, a BAG nucleic acid molecule is cloned into a gene targeting vector, such as (Stratagene Cloning Systems).

The invention also includes as preferred embodiments plant vectors into which a nucleic acid molecule according to the invention has been inserted. General descriptions of plant expression vectors and reporter genes can be found in Gruber, et al., “Vectors for Plant Transformation, in Methods in Plant Molecular Biology & Biotechnology” in Glich, et al., Eds. pp. 89-119, CRC Press, 1993. Moreover, GUS expression vectors and GUS gene cassettes are available from Clontech Laboratories, Inc. (Palo Alto, Calif.), while GFP expression vectors and GFP gene cassettes are available from Aurora Biosciences (San Diego, Calif.).

The introduction of a vector into various cells, such as bacterial, yeast, insect, mammalian, and plant cells, are well known. For example, a vector can be transformed into a bacterial cell by heat shock, electroporation, or any other suitable technique. Transformation of yeast cells with a vector according to the invention may also be carried out by electroporation, for example. Methods for introduction of vectors into animal cells include calcium phosphate precipitation, electroporation, dextran-mediated transfection, liposome encapsulation, nucleus microinjection, and viral or phage infection. The introduction of heterologous nucleic acid sequences into plant cells can be achieved by particle bombardment, electroporation, microinjection, and Agrobacterium-mediated gene insertion (for reviews of such techniques, see, e.g., Weissbach & Weissbach, Methods for Plant Molecular Biology, Academic Press, NY, Section VHI, pp. 421-463; 1988; Grierson & Corey, Plant Molecular Biology, 2d Ed., Blackie, London, Ch. 7-9, 1988; and Horsch, et al., Science, vol. 227:1229, 1985; and Gene Transfer to Plants, eds. Potrykus. Springer Verlaag, 1995).

### 3. Transgenic Plants and Plant Cells

As described above, a primary aspect of this invention concerns transgenic plants that are resistant to or tolerant of one more abiotic and/or biotic stresses. Representative plants that can be transformed with a gene coding for a QM gene include tomato, potato, arabidopsis, tobacco, cotton, rapeseed, field bean, soybean, pepper, lettuce, pea, alfalfa, clover, cole, cabbage, broccoli, cauliflower, Brussels sprout, radish, carrot, beet, eggplant, spinach, cucumber, squash, melon, cantaloupe, sunflower, ornamental, asparagus, corn, barley, wheat, rice, sorghum, onion, pearl millet, rye, and oat plants.

A. General Methods

Generally, a transgenic plant is generated by (a) transforming a plant cell with a nucleic acid of interest and (b) regenerating the plant cells to provide a differentiated plant. Frequently, resulting transgenic plants are examined to confirm the presence of the desired transgene. The nucleic acid of interest is usually contained in a vector. However, naked nucleic acid of interest may also be used even though only low efficiency transformation will likely occur.

1. Vectors and Expression Cassettes

Although a general discussion of vectors of this invention is provided above, the following description contains additional information specific to vectors useful in plant cell transformation. Usually, to be effective in regulating the expression, a promoter functional in the plant cells to be transformed is operably associated with a nucleic acid molecule of the invention to form an expression cassette that is carried in the vector. Additionally, a polyadenylation sequence and/or transcription control sequence, also recognized in the cells of the eukaryotic organism to be transformed, may also be included in the expression cassette in operable association with the promoter and QM protein structural gene. It is also preferred that the vector contain one or more genes encoding selectable markers so that transformed cells can easily be selected from non-transformed cells in culture.

(a) Promoters

Any promoter functional in plant cells may be used for generating transgenic plants of this invention, including constitutive, inducible/developmentally regulated, and tissue-specific promoters. Although endogenous plant promoters and QM protein-encoding gene promoters may be utilized in some embodiments, preferably the promoters are heterologous to the QM protein-encoding structural gene. Such regulatory sequences may be obtained from plants, viruses, or other sources.

Examples of constitutive promoters include the 35S RNA and 19S RNA promoters of cauliflower mosaic virus (CaMV), the promoter for the coat protein promoter to TMV (Akamatsu, et al., EMBO J. 6:307, 1987), promoters of seed storage protein genes such as Zma10Kz or Zmag12 (maize zein and glutelin genes, respectively), “housekeeping genes” that are express in some or all cells of a plant, such as Zmaact, a maize actin gene (see Benfey, et al., Science, vol. 244:174-181, 1989; Elliston in Plant Biotechnology, eds. Kung and Arntzen, Butterworth Publishers, Boston, Mass., p. 115-139, 1989), the patatin gene promoter from potato (see, e.g., Wenzier, et al., Plant Mol. Biol., vol. 12:41-45, 1989), the ubiquitin promoter (see, e.g., EP Patent Application 0342926), and the Chlorella virus DNA methyltransferase promoter (see, e.g., U.S. Pat. No. 5,563,328)

Inducible promoters are also useful in practicing the present invention. An inducible promoter is capable of directly or indirectly activating transcription of an operably associated nucleic acid molecule in response to an inducer. The inducer may be biotic or abiotic, such as an environmental signal such as light, heat, or cold, as well as in response to a protein, a metabolite (sugar, alcohol, etc.), a growth regulator, a herbicide, etc., or indirectly through the action of a pathogen or disease agent such as a virus. A plant cell containing an inducible promoter may be exposed to an inducer by externally applying the inducer to the cell such as by spraying, watering, heating, exposure to light, exposure to a pathogen, or similar methods.

To be most useful, an inducible promoter preferably provides low or no expression in the absence of the inducer; provides high expression in the presence of the inducer; and uses an induction scheme that does not interfere with the normal physiology of, for example, the plant and has little effect on the expression of other genes in the eukaryote. Examples of inducible promoters useful within the context of the present invention include those induced by chemical means, such as the yeast metallothionein promoter activated by copper ions; In2-1 and In2-2 regulator sequences activated by substituted benzenesulfonamides, e.g., herbicide safeners; the promoter sequence isolated from a 27 kD subunit of the maize glutathione-S-transferase (GST II) gene induced by N,N-diallyl-2,2-dichloroacetamide (common name: dichloramid) or benzyl-2-chloro-4-(trifluoromethyl)-5-thiazolecarboxylate (common name: flurazole); GRE regulatory sequences induced by glucocorticoids, and an alcohol dehydrogenase promoter induced by ethanol. Other inducible promoters include those induced by pathogen attack (see, e.g., U.S. Pat. No. 6,100,451), a chalcone synthase promoter, and the defense activated promoter (prop1-1) (Strittmatter, et al., Bio/Technology, vol. 13:1085-1089, 1995). Inducible promoters also the inducible promoters from the PR protein genes, especially the tobacco PR protein genes, such as PR-1a, PR-1b, PR-1c, PR-1, PR-A, PR-S, the cucumber chitinase gene, and the acidic and basic tobacco beta-1,3-glucanase genes. Wound inducible (WIN) promoters may also be useful in the context of the present invention.

Tissue-specific promoters may also be utilized. Specific examples of tissue-specific promoter include shoot meristem-specific promoters; the tuber-directed class I patatin promoter; promoters associated with potato tuber ADPGPP genes; the seed-specific promoter of beta-conglycinin, also known as the 7S protein; seed-specific promoters from maize zein genes; pollen-specific promoters (see, e.g., U.S. Pat. Nos. 5,086,169 and 5,412,085); an anther-specific promoter (see, e.g., U.S. Pat. No. 5,477,002); and a tapetum-specific promoter (see, e.g., U.S. Pat. No. 5,470,359).

Promoters that drive gene expression based on developmental stage or temporally may also be used.

(b) Markers

The vectors of the present invention, also preferably include at least one selectable or scorable marker/reporter that is functional in eukaryotic cells of the type to be transformed, for example, plant cells. A selectable marker gene includes any gene that confers a phenotype or trait on the host cells that allows transformed cells to be identified and selectively grown. Accordingly, the selection marker genes may encode polypeptides that confer on transformed cells resistance to a chemical agent or to physiological stress, or a distinguishable phenotypic characteristic to the cells such that cells transformed with the recombinant nucleic acid molecule may be easily selected using a selective agent. Specific examples for the genes suitable for this purpose have been identified may be found in, for example, Fraley, in Plant Biotechnology, eds. Kung and Amtzen, Butterworth Publishers, Boston, Mass., p. 395-407, 1989, and in Weising, et al., Ann. Rev. Genet., vol. 22:421-77, 1988.

2. Transformation

Transformation can be accomplished by any suitable method. Plant cell transformation may be carried out using any suitable technique for introducing nucleic acids into plant cells. See, e.g., Methods of Enzymology, vol. 153, 1987, Wu and Grossman, Eds., Academic Press). Herein, “transformation” means alteration of the genotype of cell by the introduction of one or more heterologous nucleic acid molecules. Transformation may be either transient or permanent, with permanent genetic alteration being preferred.

Methods of introducing vectors into monocotyledenous or dicotyledenous plant cells include physical and/or chemical means, such as electroporation, microinjection into plant cell protoplasts, particle bombardment, and viral and bacterial infection/co-cultivation. and are applicable to both monocotyledenous and dicotyledenous plants. The principle methods of causing stable integration of exogenous DNA into plant genomic DNA include the following approaches: Agrobacterium-mediated gene transfer; direct DNA uptake, including methods for direct uptake of DNA into protoplasts; DNA uptake induced by brief electric shock of plant cells; DNA injection into plant cells or tissues by particle bombardment; by the use of micropipette systems; and by the direct incubation of DNA with germinating pollen; and the use of viral vectors. As those in the art will appreciate, the particular transformation method chosen will depend on many factors, including the eukaryote species to be transformed, but in any event is a matter of routine.

In the context of complex multicellular organisms such as plants, it may be useful to generate a number of individual transformed plants with any recombinant construct in order to recover plants free from any effects related to the position in which the expression cassette becomes integrated. In certain embodiments it may be preferable to select plants that contain one copy of the introduced nucleic acid molecule, while in other embodiments, multiple copies of the expression may be preferred.

In particularly preferred embodiments, the Agrobacterium Ti plasmid system is utilized to perform plant cell transformation. The tumor-inducing (Ti) plasmids of A. tumefaciens contain a segment of plasmid DNA known as transforming DNA (T-DNA) that is transferred to plant cells where it integrates into the plant host genome. The construction of the transformation vector system typically has two basic steps. First, a plasmid vector is constructed that replicates in E. coli. This plasmid contains an expression cassette capable of directing the expression of a DNA molecule according to the invention (e.g., a DNA having a nucleotide sequence of SEQ ID NO: 1 or 2) flanked by T-DNA border sequences that define the points at which the DNA integrates into the plant genome. Usually a gene encoding a selectable marker (such as a gene encoding resistance to an antibiotic such as Kanamycin) is also inserted between the left border (LB) and right border (RB) sequences. The expression of this gene in transformed plant cells allows for positive selection of plant cells that contain an integrated T-DNA region. The second step entails transfer of the plasmid from E. coli to Agrobacterium. This can be accomplished via a conjugation mating system, or by direct uptake of plasmid DNA by Agrobacterium. For subsequent transfer of the T-DNA to plants, the Agrobacterium strain utilized contains a virulence (vir) genes for T-DNA transfer to plant cells. Those skilled in the art recognize that there are multiple choices of Agrobacterium strains and plasmid construction strategies that can be used to optimize genetic transformation of plants. Methods of inoculation of the plant tissue vary depending upon the plant species and the Agrobacterium delivery system. A very convenient approach is the leaf disc procedure that can be performed with any tissue explant that provides a good source for initiation of whole plant differentiation. The addition of nurse tissue may be desirable under certain conditions. Other procedures such as the in vitro transformation of regenerating protoplasts with A. tumefaciens may be followed to obtain transformed plant cells as well.

In other embodiments, transformation is accomplished using direct physical or chemical means. For example, the nucleic acid can be physically transferred by microinjection directly into plant cells by use of micropipettes or particle bombardment. Alternatively, the nucleic acid may be transferred into the plant cell by using polyethylene glycol which forms a precipitation complex with genetic material that is taken up by the cell.

Another method for introducing nucleic acid into a plant cell is high velocity ballistic penetration by small particles that either contain or are coated with the nucleic acid to be introduced (see, e.g., U.S. Pat. Nos. 4,945,050, 5,036,006, and 5,100,792). Typically, when utilizing particle bombardment, the DNA to be delivered is adsorbed on microprojectiles such as magnesium sulfate crystals or tungsten particles, and the microprojectiles are physically accelerated into cells or plant tissues.

Heterologous nucleic acid can also be introduced into plant cells by electroporation. In this technique, plant protoplasts are electroporated in the presence of vectors or expression cassettes containing a nucleic acid molecule according to the invention. Electrical impulses of high field strength reversibly permeabilize membranes allowing the introduction of the nucleic acids into the plant cells. Electroporated plant protoplasts reform cell walls, divide, and form callus tissue. Selection of transformed plant cells can be accomplished using any suitable technique.

After selecting transformed cells, expression of the desired QM-protein-encoding gene can be confirmed. For example, simple detection of RNA transcribed from the inserted DNA can be achieved by well-known methods in the art, such as Northern blot analysis. Alternatively, the inserted sequence can be identified, for example, using the polymerase chain reaction and Southern blot analysis. Expression levels and copy number can also be assessed using well-known techniques.

3. Regeneration of Transgenic Plants

Transformed plant cells that express a desired QM protein species can be regenerated into a whole plant using any known technique. Here, “regeneration” refers to growing a whole plant from a transformed protoplast, a plant cell, a group of plant cells (e.g., plant callus), a plant tissue, or a plant organ or part.

Regeneration from protoplasts varies from species to species of plants, but generally a suspension of protoplasts is first made. In certain species, embryo formation can then be induced from the protoplast suspension, to the stage of ripening and germination as natural embryos. The culture media generally contains various amino acids and hormones necessary for growth and regeneration. Examples of hormones utilized include auxin and cytokinins. It is sometimes advantageous to add glutamic acid and proline to the medium, especially for such species as corn and alfalfa. Efficient regeneration depends on many variables, including the medium used, the genotype of the plant cells, and the history of the culture.

Regeneration also occurs from plant callus, tissues, organs, or parts. Transformation can be performed in the context of organ or plant part regeneration (see, e.g., Methods in Enzymology, vol. 118, and Klee, et al., Ann. Rev. Plant Phys., vol. 38:467, 1987). Utilizing a leaf disk-transformation-regeneration method (see, e.g., Horsch, et al., Science, vol. 227:1229, 1985), disks are cultured on selective media, followed by shoot formation in about 2-4 weeks. Shoots that develop are excised from calli and transplanted to appropriate root-inducing selective medium. Appropriate selection media are known in the art (see, e.g., Curry and Cassells in: Plant Cell Culture Protocols, pp. 31-43, Humana Press, Totowa, N.J., 1999; Blackwell et al, IBID 19-30, 1999; Franklin and Dixon in: Plant Cell Culture, pp. 1-25, IRL Press, Oxford, 1994). Rooted plantlets are transplanted to soil as soon as possible after roots appear. The plantlets can be repotted, as required, until reaching maturity.

Regeneration also occurs from plant callus, tissues, organs, or parts. Transformation can be performed in the context of organ or plant part regeneration (see, e.g., Methods in Enzymology, vol. 118, and Klee, et al., Ann. Rev. Plant Phys., vol. 38:467, 1987). Utilizing a leaf disk-transformation-regeneration method (see, e.g., Horsch, et al., Science, vol. 227:1229, 1985), disks are cultured on selective media, followed by shoot formation in about 2-4 weeks. Shoots that develop are excised from calli and transplanted to appropriate root-inducing selective medium. Appropriate selection media are known in the art (see, e.g., Curry and Cassells in: Plant Cell Culture Protocols, pp. 31-43, Humana Press, Totowa, N.J., 1999; Blackwell et al, IBID 19-30, 1999; Franklin and Dixon in: Plant Cell Culture, pp. 1-25, IRL Press, Oxford, 1994). Rooted plantlets are transplanted to soil as soon as possible after roots appear. The plantlets can be repotted, as required, until reaching maturity.

Parts obtained from the transgenic plant, such as flowers, seeds, leaves, branches, fruit, and the like, are included in the invention. As will be appreciated, in some vegetatively propagated plant species, the root portion may be transgenic (i.e., be engineered to contain a nucleic acid molecule according to the invention), while the upper portion of the plant may not be. Alternatively, the portion of the plant grafted onto the root stock may be transgenic (i.e., be engineered to contain a nucleic acid molecule according to the invention), while the root stock may not be. In other embodiments, both the root stock and vegetatively propagated portions are transgenic. Progeny and variants, and mutants of the regenerated plants are also included within the scope of the present invention, provided that these parts comprise the introduced heterologous nucleic acid sequences.

B. Generation of Transgenic Organisms with Desirable Traits

Transgenic eukaryotic organisms and cells of the invention, particular plants and yeast and cell lines used for bioproduction, are resistant to, or tolerant of, biotic and abiotic stresses. Additionally, they exhibit delayed senescence.

Biotic stresses result directly or indirectly from a challenge by a biotic agent. Biotic agents include insects, fungi, bacteria, viruses, nematodes, viroids, mycloplasmas, etc. Biotic agents typically induce programmed cell death in affected plant cells. Such programmed cell death is thought to occur to inhibit the spread of an invading pathogen. However, the transgenic plants of the invention have exhibited resistance to a variety of biotic agents, including pathogens such as fungi and viruses. An exemplary pathogen is the fungal pathogen Sclerotinia sclerotiorum, which is one of the most nonspecific and omnivorous plant pathogens known. Further, a variety of other economically important pathogens are known, including the fungi Botrytis cinerea, Magnaportyhe grisea, Phytophthora spp, Cochliobolus spp, Fusarium graminearum and other Fusarium spp, nemtodes (such as the Meloidogyne, or “root knot”, nematodes), viruses such as tobacco mosaic virus (TMV) and tomato spotted wilt virus (TSWV), tobacco etch virus (TEV), tobacco necrosis virus (TNV), wheat streak mosaic virus (WSMV), soil borne wheat mosaic virus (SBWMV), barley yellow dwarf virus (BYDV), bacteria such as various Pseudomonas and Xanthomonas species, as well as many others.

Abiotic stress can be caused, for example, various environmental factors, such as drought, flooding) nutrient deficiency, radiation levels, air pollution, heat shock, cold shock, and soil toxicity, as well as herbicide damage, pesticide damage, or other agricultural practices. Accordingly, given that such abiotic agents play an increasing role in the viability of a variety of plant types including, food crops and ornamentals, the present invention can be utilized to produce plants or plant products (e.g., fruits, vegetables, seeds, flowers, etc.) with increased resistance to stresses such as these. Indeed, transgenic plants and plant products according to the invention are resistant to, or tolerant of, a plurality of such stresses, whether encountered simultaneously or at different times. As a result, the transgenic plants of the invention may be cultivated in new areas, thereby increasing the growth range for particular species or variety. In addition, because the transgenic plants of the invention are more tolerant to the range of growth conditions encountered in the cultivation of commercially relevant plant varieties, fewer plant varieties may be required over an existing, or even increase growth range. Similarly, improved stress resistance and tolerance will lead to increased yields of desired plant products under a variety of conditions.

One skilled in the art will readily recognize that given the disclosure provided herein, resistance to a particular biotic or abiotic stress, or combination of stresses, can be easily tested using whole plant or leaf sections, as appropriate. For example, a plant leaf may be inoculated with virus and lesion development and expansion may be measured at different time intervals. In another example, whole transgenic plants may be subjected to an abiotic stress such as high or low temperature. Stress responses, survival rates, etc. may be measured and compared to wild-type controls.

Senescence in plants is known to be a regulated process that ultimately results in cell death. Further, it is accompanied by many biochemical and structural changes, such as induction of cysteine proteases, RNases, etc., consistent with PCD. Inhibiting or delaying senescence can lead to longer shelf-lives for plant products, including fruits, vegetables, and flowers, as well as leading to increased longevity and aesthetic appeal of cut flowers and other ornamentals. In addition, in living plants increased flowering duration and fruit production may be achieved. Accordingly, the present invention has wide utility in both the food stuff market as well as the ornamental market.

Any known method for assessing senescence in plants or plant cells, tissues, or products may be used to test for decreased or delayed senescence. Such methods include, for example, characterization of fruit ripening processes, measurement of flower life, and detection of ethylene production (see, e.g., U.S. Pat. No. 5,702,933; Ryu, et al., Proc. Natl. Acad. Sci. USA, vol. 94:12717-21, 1997).

### 4. Methods of Modulating Apoptosis

The invention also provides methods for modulating apoptosis in eukaryotic cells or organisms transformed to exhibit altered QM protein expression. Taking plants as an example, generally such methods comprise generating a transgenic plant according to the invention and then identifying a transformed plant that, as compared to a wild-type plant of the same variety, exhibits an altered apoptotic response upon exposure to a biotic or abiotic stress, or combination of stresses. Any known method for assaying apoptosis may be used in this regard. For instance, a transformed plant or a portion thereof the plant may be challenged with a biotic or abiotic agent, after which the morphology of the inoculation site can observed for apoptotic signs. Alternatively, or in addition, cells or tissue from the inoculation site(s), as well as surrounding cells and tissues, if desired, can be further characterized by subsequent analysis for DNA fragmentation (e.g., by agarose gel electrophoresis), nuclear condensation (e.g., by Hoechst or DAPI staining), the change of the number of TUNEL-positive cells compared to control samples, etc.

### EXAMPLES

The following Examples are provided to illustrate certain aspects of the present invention and to aid those of skill in the art in practicing the invention. These Examples are in no way to be considered to limit the scope of the invention in any manner.

### Example 1

### Yeast Strains and Culture

Saccharomyces cerevisiae strains EGY48 (MATα his 3 trp1 ura3 LexAop-Leu2; Clontech, Inc., Mountain View, Calif.) and C15-1A (put1; MATa ura3-52 trp2 put1-54) were used as the wild-type and mutant strains, respectively, described in the Examples below. Yeast strains were routinely cultured in YPD (1% yeast extract, 2% peptone, 2% dextrose) or synthetic dropout (SD) media with appropriate supplements at 30° C. When indicated, 1.6 mM proline was added to the SD medium.

### Example 2

### Plasmid Construction and Yeast Transformation

A Put1p expression vector was made by subcloning the PUT1 gene (Wang and Brandriss (1986), Mol. Cell. Biol., vol. 6:2638-2645) by PCR into a pYES2 shuttle vector (Invitrogen, Carlsbad, Calif.) for expression from the GAL1 promoter as a C-terminal six-His tag fusion protein. A construct of the PUT1 gene also was designed that lacked the mitochondrial signaling peptide (pYES2-Put1p18). Computational analysis (MitoProt II; Institute of Human Genetics, Technical University Munich/GSF National Research Center, Germany) of the Put1p primary structure predicts that the first 18 residues are involved in mitochondrial signaling. The pYES2-Put1p18 construct was made from pYES2-PUT1 by using QuikChange (Stratagene, La Jolla, Calif.) site-directed mutagenesis.

The fusion constructs pLexA-Bax and pLexA-PUT1 were prepared by amplifying DNA fragments (EcoRI-XhoI) of the PUT1 gene and the mammalian proapoptotic bax gene by PCR and ligating the fragments into the yeast expression vector pLexA. The structures of the above constructs were confirmed by nucleic acid sequencing. Constructs were transformed into the yeast strain EGY48 (wild type) using lithium acetate. Transformed cells were plated on SD/Glu/-His medium, where expression of PUT1 or the bax gene was repressed by glucose. To induce expression of Put1p and the lethal effect of Bax, the transformed cells were plated on SD/Gal/Raff/-His medium, where expression of the PUT1 or bax gene, respectively, was induced by galactose.

Synthesis of Put1p and Put1p18 was confirmed by Western blot analysis. Put1p was overexpressed in EGY48 at 30° C. in SD/Gal medium. After harvesting, cells were resuspended in phosphate-buffered saline solution containing a protease cocktail (Sigma, St. Louis, Mo.) and broken by two freeze-thaw cycles in liquid N₂ and by a mini bead beater (BioSpec Products, Inc., Bartlesville, Okla.) with a pulse sequence of 30 s on, 60 s off, and 30 s on. Cell debris was removed by centrifugation (5 min, 4° C., 15,000×g), and the resulting cell extracts were separated by Tris-glycine sodium dodecyl sulfate-polyacrylamide gel electrophoresis and electroblotted onto an Immuno-Blot polyvinylidene difluoride membrane (0.2-μm pore size; Bio-Rad, Hercules, Calif.) with an EBU-4000 Semi-Dry electrophoretic blotting system (CBS Scientific Company, Inc., Del Mar, Calif.). Colorimetric detection of the C-terminal six-His tags of Put1p and Put1p18 was performed with a His tag monoclonal antibody kit (Novagen, San Diego, Calif.) and an enhanced chemiluminescence detection system (Pierce, Rockford, Ill.).

### Example 3

### Stress Tolerance Assays

Early-log-phase yeast cultures (optical density at 600 nm [OD₆₀₀], 0.5) were diluted to an OD₆₀₀ of 0.05 with appropriate SD medium. For chemical treatment, selected concentrations of H₂O₂ were added to the culture and incubated at 30° C. with vigorous shaking for 6 h. For heat stress, yeast cells were incubated at 50° C. for the indicated times. Following these treatments, cell viability was determined by counting the number of CFU. Ten-microliter aliquots of cell culture were diluted and spread on YPD plates and then incubated at 30° C. for 48 h. The number of CFU from treated cells was compared to the number of CFU from untreated cells. All experiments were repeated at least three times. To evaluate yeast cell viability, yeast strains were grown in appropriate SD medium overnight and then evaluated in a spot assay. The cells were serially diluted fivefold, and 5-μl aliquots from each dilution were spotted onto SD/Glu or SD/Gal medium, incubated at 30° C. for 3 days, and photographed.

Paraquat, a contact herbicide, uncouples electron transport and generates high levels of superoxide that are lethal to EGY48. Sensitivity to paraquat is decreased following treatment with exogenous proline. The Put1p-overexpressing strain (with lower proline levels), pYES2-PUT1, and pYES2-Put1p18, the put1-deficient strain (with higher proline levels), were assayed to determine if paraquat sensitivity was related to proline metabolism. Put1p-overexpressing yeast cells were found to be more sensitive to oxidants such as paraquat and H₂O₂ than the control transformants. Similar results were obtained with a pYES2-PUT1 construct in EGY48, demonstrating that the Put1p fusion product from the pLexA-PUT1 construct was not contributing to the oxidative stress sensitivity. The put1-disrupted strain had more protection from these stresses. Thus, overexpression of Put1p, which should lower intracellular proline levels, reduces the protection of cells from oxidative stress and cell death.

Proline catabolism occurs in the mitochondria, where Put1p couples the oxidation of proline to the reduction of the electron transport system. Overexpression of an N-terminal deletion construct (pYES2-Put1p18) that lacks the mitochondrial signaling peptide did not affect the oxidative stress sensitivity, indicating that mislocalization of Put1p prevents its proper function in proline catabolism.

Paraquat, a contact herbicide, uncouples electron transport and generates high levels of superoxide that are lethal to EGY48. Sensitivity to paraquat is decreased following treatment with exogenous proline. The Put1p-overexpressing strain (with lower proline levels), pYES2-PUT1, and pYES2-Put1p18, the put1-deficient strain (with higher proline levels), were assayed to determine if paraquat sensitivity was related to proline metabolism. Put1p-overexpressing yeast cells were found to be more sensitive to oxidants such as paraquat and H₂O₂ than the control transformant. Similar results were obtained with a pYES2-PUT1 construct in EGY48, demonstrating that the Put1p fusion product from the pLexA-PUT1 construct was not contributing to the oxidative stress sensitivity. The put1-disrupted strain had more protection from these stresses. Thus, overexpression of Put1p, which lowers intracellular proline levels, reduced the protection of cells from oxidative stress and cell death.

### Example 4

### Intracellular Proline Levels

Yeast cells were inoculated in SD medium supplemented with 2% galactose and 1% raffinose and incubated for 3 days at 30° C. Following stress treatment, 5 ml of cell suspension was removed, washed twice with 0.9% NaCl, and suspended in 0.5 ml of distilled water. The cells were transferred to a boiling water bath, and intracellular amino acids were extracted by boiling for 10 min. After centrifugation (5 min, 4° C., 15,000×g), the supernatant was free of proteins. Intracellular proline levels were assayed by incubating 200 μl of the supernatant with 200 μl of acid-ninhydrin (0.25 g ninhydrin dissolved in 6 ml glacial acetic acid and 4 ml 6 M phosphoric acid) and 200 μl of glacial acetic acid for 1 h at 100° C. Reaction were stopped by incubation on ice, and the mixtures extracted with 400 μl toluene. The toluene phase was separated, and the OD₅₂₀ was used to determine the concentration of proline in the extract.

Proline catabolism occurs in the mitochondria, where Put1p couples the oxidation of proline to the reduction of the electron transport system. Overexpression of an N-terminal deletion construct (pYES2-Put1p18) that lacks the mitochondrial signaling peptide did not affect the oxidative stress sensitivity, indicating that mislocalization of Put1p prevents its proper function in proline catabolism.

Proline was measured in Put1p-overexpressing yeast cells, pull mutant cells, and control wild-type cells, as shown in Table 1, below.

TABLE 1
Intracellular proline levels in yeast

| | Proline Levels (mM) | | | | |
|---|---|---|---|---|---|
| Treatment | Vector | PUTΔ | PUT1 | PUT1 + tQM | tQM |
| No stress | 3.15 ± 0.30 | 5.22 ± 0.37 | 0.51 ± 0.03 | 3.34 ± 0.32 | 2.46 ± 0.15 |
| H₂O₂ (6 h) | 2.40 ± 0.05 | 6.35 ± 0.55 | 0.62 + 0.08 | 4.35 + 0.40 | 4.31 + 0.42 |
| 50° C. (4 h) | 1.05 ± 0.05 | 4.81 ± 0.30 | 0.23 ± 0.01 | 5.34 ± 0.48 | 3.50 ± 0.55 |

The Put1p-overexpressing strain had the lowest levels of proline, while the put1 disruptant yeast strain accumulated the highest levels of proline. Thus, higher proline levels are correlated with increased protection from oxidative stress in yeast.

### Example 5

### Intracellular ROS Levels

The production of ROS by yeast cells was detected by incubating 10⁶ to 10⁷ cells in 500 μl SD/Gal medium with 50 μM dihydrorhodamine 123 (DHR123; Molecular Probes, Eugene, Oreg.) for 15 min at room temperature. The samples were viewed with a fluorescent microscope equipped with a rhodamine optical filter. ROS levels were quantified by measuring the green fluorescence intensity of rhodamine-123 from microscopic images with Photoshop software (Adobe Systems, Mountain View, Calif.).

Sensitivity to H₂O₂ in liquid medium was also measured. Strains overexpressing Put1p 2 and 4 hours after the addition of 3 mM H₂O₂ were more sensitive to H₂O₂ than control cells with vector alone (6% versus 69% viability and 2% versus 49% viability at 2 and 4 h, respectively). Accordingly, the put1 disruptant strain was more resistant to H₂O₂, with cell viabilities of 87% and 83% at 2 and 4 h, respectively.

### Example 6

### Conditional Life/Death Yeast Screen

A tomato cDNA library, constructed from tobacco mosaic virus-infected tomato VF36 leaves, was cloned into the yeast expression vector pB42AD and transformed into EGY48 overexpressing Put1p. The transformed cells were plated on SD/Glu/-His/-Trp medium, where the expression of genes in the library was repressed by glucose. Growing cells were collected, washed, and plated on SD/Gal/Raf/-His/-Trp medium, where the expression of genes in the library was induced by galactose supplemented with 2 mM paraquat. From 5×10⁶ transformants, hundreds of colonies were recovered. 95 colonies were arbitrarily selected and reinoculated onto paraquat-containing plates. After a 5-day incubation at 30° C., the 14 surviving colonies were again streaked onto SD/Gal/Raf/-His/-Trp plates supplemented with 2 mM paraquat to confirm the resistance phenotype. Six of them contained tomato cDNA that when reintroduced to the Put1p-overexpressing EGY48 strain again conferred paraquat resistance when grown on paraquat-containing SD/Gal medium, confirming the resistance phenotype. One of the clones encoded a predicted QM-like protein comprised of 179 amino acids and having an estimated molecular mass of 20.3 kDa. The isolated cDNA (tQM) was expressed under the control of a constitutive GAL1 promoter, and the resultant plasmid, pB42AD-tQM, was used for further analysis. Sequence alignment with several QM homologues revealed that the tQM protein shares significant sequence identity with those proteins in the alignment region(s), including S. cerevisiae GRC5 (73% sequence identity) and human ribosomal protein L10 (77% sequence identity).

EGY48 was transformed with pB42AD, pLexA-PUT1, pLexA-PUT1 plus pB42AD-tQM, and pB42AD-tQM and grown on SD medium supplemented with 2 mM paraquat. Expression of tQM enabled the Put1p-overexpressing strain to grow and survive on medium amended with lethal levels of paraquat. Growth was similar on media with and without paraquat. Similar results were obtained with H₂O₂ treatment, indicating that tQM can rescue yeast from oxidative stress.

### Example 7

### Heat Stress Resistance Conferred by tQM

ROS levels increase in yeast cells subjected to heat stress (50° C.), and ROS production is directly involved in heat-induced cell death. Yeast strains were incubated at 50° C. for 4 h, and culture aliquots were spotted on YPD. The Put1p-overexpressing strain was very sensitive to heat stress, but transformants expressing tQM exhibited reduced heat sensitivity, further supporting that QM proteins act as stress suppressors in response to various oxidative stresses.

Before and after heat treatment, fluorescence emission by the ROS indicator DHR123 was much higher in the Put1p-overexpressing strain (425% versus 650%) than in the control (100% versus 440%) or the tQM-overexpressing strains (20% versus 150%). tQM quenched intracellular ROS generation when coexpressed with Put1p (18% versus 240%), indicating that tQM acts by scavenging heat-induced ROS.

Intracellular proline levels also were determined in these strains following treatment with H₂O₂ or heat stress. The Put1p-overexpressing strain has the lowest proline levels relative to the other yeast cells (see Table 1, above). Expression of tQM increased proline accumulation in both the wild-type EGY48 strain and the strain carrying Put1p. Stress treatment did not alter the levels of intracellular proline in the tQM yeast strain, but the proline concentration in EGY48 decreased after stress. Thus, higher proline levels are correlated with tQM expression and lower intracellular ROS levels.

### Example 8

### tQM Interaction with Put1p

The interaction between tQM and Put1p was tested in a yeast two-hybrid system. Put1p was expressed as a fusion to the LexA DNA binding domain (LexA:PUT1) and tQM was expressed as a fusion to the B42 activation domain (AD:tQM). Expression of both constructs was under control of the GAL promoter. When EGY48 was transformed with LexA:PUT1 and AD:tQM, the transformants grew on Leu⁻ selection medium and turned blue on SD/Gal medium containing 5-bromo-4-chloro-3-indolyl-d-galactoside (X-Gal) but not on SD/Glu medium. A control strain containing LexA or AD alone showed no growth on Leu⁻ medium, and no blue color was observed on X-Gal-containing medium. These results indicate that in vitro, tQM interacts directly with Put1p.

### Example 9

### Effects tQM on Bax-induced Lethality

Bax is a proapoptotic member of the Bcl-2 family of proteins. It is lethal when expressed in yeast, and its expression can be induced by ROS production. To further extend the generalization that QM proteins can function as general stress protection components, the effects of tQM expression on Bax-mediated cell death in yeast were evaluated. Yeast strains were transformed with pLexA-Bax and pB42AD-tQM, cultured in SD-glucose medium for 1 day, and spotted on SD-galactose medium. After a 5-day incubation, yeast colonies transformed with tQM and Bax were visible, but there were no colonies expressing only Bax. After a 24 h incubation on SD-galactose medium, about 60% of the cells died in the Bax-expressing strain, but in a strain coexpressing tQM, only 14% of the cells died. Expression of tQM also significantly inhibited Bax-induced ROS production as measured with DHR123. Thus, QM proteins reduce intracellular ROS levels and cell death attributable to Bax-induced oxidative stress.

### Discussion of Examples 1-9

Proline is known to be a compatible osmolyte and osmoprotectant that can also play other roles, especially with respect to its ability to scavenge free radicals, control redox homeostasis, and ameliorate transitions in redox potential by replenishing NADP⁺. When organisms are exposed to abiotic stresses, such as salt, heat, drought, cold, and UV light, ROS are produced and proline levels increase. Thus, controlling intracellular proline accumulation confers protection to divergent stresses that all generate ROS and is consistent with previous reports that proline can scavenge ROS. A dominant active Ras mutant in the phytopathogenic fungus Colletotrichum trifolii, when grown on minimal medium, produced high levels of ROS resulting in aberrant hyphal morphology and ultimately apoptotic-like programmed cell death. Exogenous proline sufficed to restore wild-type hyphal morphology by inhibiting ROS-induced programmed cell death. The ROS scavenging property of proline also protects fungal cells against other stresses, such as UV light, heat, salt, and H₂O₂. Exogenous proline also can protect S. cerevisiae cells from paraquat-mediated lethality. These results, obtained using exogenous proline supplements, demonstrate that proline acts as an antioxidant cytoprotectant during stress by scavenging ROS and maintaining redox homeostasis.

The examples above describe the manipulation of endogenous proline levels by altering the activity of Put1p, which catalyzes the rate-limiting step of proline degradation. Overexpression of Put1p reduced intracellular proline levels and increased sensitivity to oxidative stress. In put1 mutants, that lack Put1p, the accumulated intracellular proline protects the cell from oxidative stress. These observations are consistent with those in animals and plants where changes in intracellular proline levels are directly associated with stress tolerance. For example, overexpression of human proline dehydrogenase (PRODH2) can induce apoptosis in human tumor cell lines, establishing a direct correlation between reduction of proline levels and loss of viability. Moreover, suppression of proline degradation via the expression of an antisense proline dehydrogenase (AtProDH) gene in Arabidopsis thaliana improves tolerance to freezing and high salinity. These results show that control of intracellular proline levels is critical for stress tolerance.

Genes encoding the enzymes associated with the biosynthesis and degradation of proline have been cloned and partially characterized in organisms ranging from bacteria to fungi, plants, and mammals. However, the factors regulating the expression and activities of these enzymes are less well understood. The examples above describe the identification and characterization of a tomato QM-like protein, which can induce accumulation of intracellular proline and protect eukaryotic cells against oxidative stress. In plants and animals, QM also has been reported to play a role in development and/or proliferation, and may act as a tumor suppressor. A phenotypic analysis of an S. cerevisiae mutant deficient in GRC5, a homologue of QM, reported that QM is involved in multiple cellular functions, including growth control and proliferation, cytoskeletal function, and energy metabolism; however, there was no direct evidence on the mechanism through which QM regulated cell growth and development. The examples above reveal a link between QM expression, proline accumulation, and stress tolerance, as tQM directly affected Put1p function and results from the yeast two-hybrid analysis showed a physical interaction between tQM and Put1p.

The discovery that tQM could suppress Bax-induced cell death was unexpected. Bax is a proapoptotic member of the Bcl-2 gene family. While S. cerevisiae does not contain endogenous Bcl-2 family members, the initial events underlying Bax activity in yeast and mammalian cells are similar. Several mammalian proteins, including Bcl-2 and Bcl-xL, can suppress Bax-induced cell death in yeast, as they do in animal cells. Plant genes from Arabidopsis, e.g., the Bax inhibitor 1 gene (AtBI-1), and tomatoes, e.g., LePHGPx (which encodes a phospholipid hydroperoxide glutathione peroxidase), also can suppress Bax-induced lethality and protect yeast against H₂O₂ and heat stress. In the studies above, it was found that an ectopically expressed tQM protein could suppress the lethal activity of a mammalian protein Bax in yeast and that inhibition of Bax-induced cell death was correlated with reduced ROS generation. The downstream effectors of Bax-induced cell death in yeast are not yet known, but there is evidence that ROS produced in mitochondria may be included. Thus, the results above are consistent with tQM interacting with endogenous yeast proteins that are downstream of Bax in a ROS-mediated signaling pathway.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the spirit and scope of the invention. More specifically, it will be apparent that various genetic constructs can be generated that will include encode a QM protein, derivative, or variant and that will achieve the same or similar results. All such similar substitutes and modifications apparent to those skilled in the art are deemed to be within the spirit and scope of the invention as defined by the appended claims.

All patents, patent applications, and publications mentioned in the specification are indicative of the levels of those of ordinary skill in the art to which the invention pertains. All patents, patent applications, and publications are herein incorporated by reference in their entirety for all purposes and to the same extent as if each individual publication was specifically and individually indicated to be incorporated by reference.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A transgenic eukaryotic organism that exhibits altered expression of a QM protein-encoding gene.

2. A transgenic eukaryotic organism according to claim 1, wherein the altered expression of a QM protein-encoding gene results from a genetic manipulation selected from the group consisting of introduction of a foreign QM gene, duplication of an endogenous QM protein-encoding gene, and alteration of a regulatory sequence of an endogenous QM protein-encoding gene.

3. A transgenic eukaryotic organism according to claim 1, wherein the altered expression of a QM protein-encoding gene results from expression of a heterologous QM protein-encoding gene.

4. A transgenic eukaryotic organism according to claim 3, wherein expression of the heterologous QM protein-encoding gene is regulated by a promoter selected from the group consisting of a constitutive promoter and an inducible promoter.

5. A transgenic eukaryotic organism according to claim 4, wherein the promoter is a stress inducible promoter.

6. A transgenic eukaryotic organism according to claim 1 that is a multicellular organism.

7. A transgenic eukaryotic organism according to claim 6, wherein the multicellular organism is a plant.

8. A transgenic eukaryotic organism according to claim 6, wherein the plant is selected from the group consisting of a tomato, potato, arabidopsis, tobacco, cotton, rapeseed, field bean, soybean, pepper, lettuce, pea, alfalfa, clover, cole, cabbage, broccoli, cauliflower, Brussels sprout, radish, carrot, beet, eggplant, spinach, cucumber, squash, melon, cantaloupe, sunflower, ornamental, asparagus, corn, barley, wheat, rice, sorghum, onion, pearl millet, rye, and oat plant.

9. A transgenic eukaryotic organism according to claim 1 that is a single cell.

10. A transgenic eukaryotic organism according to claim 1 that is resistant to or tolerant of at least one of an abiotic stress or a biotic stress.

11. A method of producing a stress resistant transgenic eukaryotic organism, comprising transforming a eukaryotic cell with a nucleic acid molecule that encodes a heterologous QM protein-encoding gene.
